(19) United States
(12) Patent Application Publication
(10) Pub. No.: US 2024/0016884 A1
(43) Pub. Date: Jan. 18, 2024

(54) WS635 USES THEREOF IN MEDICINE

(71) Applicants: WATERSTONE PHARMACEUTICALS (WUHAN) CO., LTD., Wuhan (CN); THE GENERAL HOSPITAL CORPORATION, Boston, MA (US)

(72) Inventors: Faming ZHANG, Wuhan (CN); Yuan SHEN, Wuhan (CN); Jian CUI, Wuhan (CN); Yao YU, Wuhan (CN); Minglong HU, Wuhan (CN); Zhongcong XIE, Andover, MA (US)

(21) Appl. No.: 18/472,275

(22) Filed: Sep. 22, 2023

Related U.S. Application Data
(63) Continuation of application No. PCT/CN2021/086129, filed on Apr. 9, 2021.

(51) Int. Cl. A61K 38/12; A61P 25/28

(52) U.S. Cl. CPC A61K 38/12; A61P 25/28

(57) ABSTRACT

The present invention relates to use of the compound having Formula (I) or a stereoisomer, a tautomer, an N-oxide, a solvate, a metabolite, a pharmaceutically acceptable salt or a prodrug thereof in the manufacture of a medicament or pharmaceutical composition for preventing, treating or lessening POD,

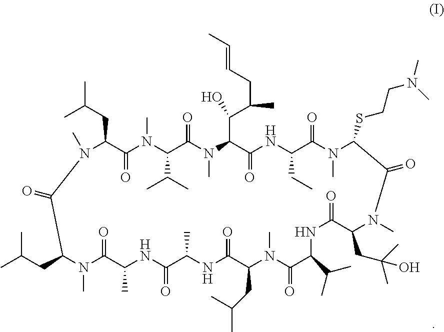 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

### CROSS-REFERENCE TO RELATED APPLICATIONS

The present application is a continuation of International Application No. PCT/CN2021/086129, filed on Apr. 9, 2021, which is hereby incorporated by reference in its entirety.

### FIELD OF THE DISCLOSURE

The present invention belongs to the field of medicine. Specifically, it relates to the of uses (3 S,6 S,9S,12R,15 S,18 S,21 S,24S,27R,30S,33 S)-27-((2-(dimethylamino)ethyl)thio)-30-ethyl-33-((1R,2R,E)-1-hydroxy-2-methylhex-4-en-1-yl)-24-(2-hydroxy-2-methy 1propyl)-6,9,18-tri isobutyl-3,21-diisopropyl-1,4,7,10,12,15,19,25,28-nonamethyl-1,4,7,10,13,16,19,22,25,28,3 1-undecaazacyclotritriacontan-2,5,8,11,14,17,20,23,26,29,32-undecaone (I) (W5635) and pharmaceutically compositions thereof in the manufacture of a medicament for preventing, managing, treating or lessening Postoperative Delirium (POD) in a patient.

### BACKGROUND OF THE INVENTION

WS635 (the compound of formula (I)) is a novel non-immunosuppressive cyclosporine-based analog that exhibits potent suppression of hepatitis C virus (HCV) replication in vitro. WS635 inhibited the peptidyl prolyl isomerase activity of cyclophilin A at nanomolar concentrations but showed no detectable inhibition of calcineurin phosphatase activity at concentrations up to 2 μM. Metabolic studies indicated that WS635 did not induce the major cytochrome P450 enzymes 1A2, 2B6, and 3A4. WS635 was a weak inhibitor and a poor substrate for P-glycoprotein. Functional assays with stimulated Jurkat cells and stimulated human peripheral blood mononuclear cells indicated that WS635 is a weaker inhibitor of interleukin-2 secretion than cyclosporine. A series of two-drug combination studies was performed in vitro. WS635 exhibited synergistic antiviral activity with alpha interferon 2b and additive antiviral activity with ribavirin. WS635 was shown to be orally bioavailable in multiple animal species and produced blood and liver concentrations of parent drug that exceeded the 50% effective dose determined in the bicistronic con1b-derived replicon assay. These results suggest that WS635 warrants further investigation as a novel therapeutic agent for the treatment of individuals who are chronically infected with HCV.

Every year, over 312 million patients worldwide have surgery under anesthesia. Postoperative Delirium (POD) is one of the most common postoperative complications in geriatric patients. They have independent adverse effects on long-term morbidity, mortality, the cost of healthcare, and quality of life.

### SUMMARY OF THE DISCLOSURE

The following just summarizes some aspects of the invention, but are not limited to these. These aspects and other parts will be described more completely later. All references of this specification are incorporated herein by reference in their entirety. Where there are differences between disclosure of the present specification and cited references, the disclosure of the present specification controls.

In the research and development process, the inventors surprisingly found that WS635 could significantly attenuate the anesthesia/surgery (1.4% isoflurane and abdominal surgery)-induced cognitive impairment in mice. The inventors further investigated that the WS635 could be used to treat Postoperative Delirium (POD).

Specifically, in one aspect, the present invention relates to use of the compound having Formula (I) or a stereoisomer, a tautomer, an N-oxide, a solvate, a metabolite, a pharmaceutically acceptable salt or a prodrug thereof in the manufacture of a medicament or pharmaceutical composition for preventing, treating or lessening Postoperative Delirium (POD),

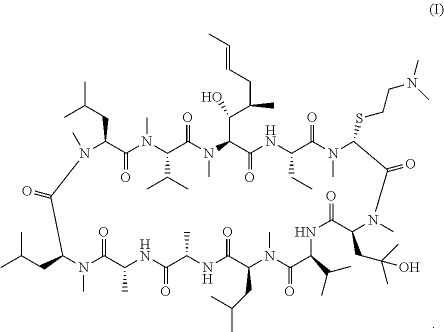 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

In the research and development process, the inventors surprisingly found that WS635 could attenuate the anesthesia/surgery-induced changes in Buried Food Test with a dose-dependent manner. Furthermore, the inventors surprisingly found that WS635 could attenuate the anesthesia/surgery-induced changes in Open Field Test and attenuate the anesthesia/surgery-induced changes in Y-maze Test. According to the examples of present disclosure, the said compound of formula (I) can attenuate the anesthesia/surgery-induced changes in the delirium-like behaviors in mice, and have amazingly good potential on preventing, treating or lessening POD.

In one embodiment, the pharmaceutical composition further comprising a pharmaceutically acceptable carrier, excipient, adjuvant or a combination thereof.

In one embodiment, the pharmaceutical composition further comprising other agent used for preventing, treating or lessening cognitive impairment.

In one embodiment, the compound is administered at a daily dose of less than about 900 mg.

In one embodiment, the compound is administered at a daily dose of between about 10 to about 900 mg.

In one embodiment, the compound is administered at a daily dose of between about 50 to about 600 mg.

In one embodiment, the compound is administered 1 time per day.

In one embodiment, the compound is administered 1 time per day as a single dosage.

In one embodiment, the compound is administered by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intraperitoneally and intrathecally.

In one embodiment, the compound is administered orally or intravenously.

In one embodiment, the compound is administered in a form of tablet, capsule or injection.

In another aspect, provided herein is a method for preventing, treating or lessening POD in a patient comprising administering to the patient therapeutically effective amount of the compound having Formula (I) or a stereoisomer, a tautomer, an N-oxide, a solvate, a metabolite, a pharmaceutically acceptable salt or a prodrug thereof. As described above, the said compound of formula (I) can attenuate the anesthesia/surgery-induced changes in delirium-like behaviors, and have amazingly good effect on preventing, treating or lessening POD. According to the examples of present disclosure, the said method can prevent, treat or lessen POD in a patient effectively.

In one embodiment, the therapeutically effective amount is 40 mg/kg in mice. The inventors found that treatment with 40 mg/kg WS635 can more effectively attenuate the anesthesia/surgery-induced changes of delirium-like behaviors in mice.

In one embodiment, the therapeutically effective amount is 4.4 mg/kg in human.

In one embodiment, the compound is administered to the subject within 1 hour prior to the operation.

In one embodiment, the compound is administered to the subject within 0.5 hour prior to the operation.

In one embodiment, the compound is administered at a daily dose of less than about 900 mg.

In one embodiment, the compound is administered at a daily dose of between about 10 to about 900 mg.

In one embodiment, the compound is administered at a daily dose of between about 50 to about 600 mg.

In one embodiment, the compound is administered 1 time per day.

In one embodiment, the compound is administered 1 time per day as a single dosage.

In one embodiment, the compound is administered by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intraperitoneally and intrathecally.

In one embodiment, the compound is administered orally, intravenously or intraperitoneally.

In one embodiment, the compound is administered in a form of tablet, capsule or injection.

In one embodiment, wherein the compound is administered in combination with one or more other agent used for preventing, treating or lessening cognitive impairment other than the compound of Formula I.

In another aspect, provided herein is a pharmaceutical composition comprising a therapeutically effective amount of a compound of Formula I or a stereoisomer, a tautomer, an N-oxide, a solvate, a metabolite, a pharmaceutically acceptable salt or a prodrug thereof, for use in treating, preventing, or lessening Postoperative Delirium (POD).

In one embodiment, the composition is formulated in a single dose form.

In one embodiment, the composition is formulated in a single dose form wherein such single dose form comprises less than 900 mg of Compound I.

In one embodiment, wherein such single dose further comprise one or more other agent used for preventing, treating or lessening cognitive impairment other than the compound of Formula I.

In another aspect, provided herein is a compound of Formula I or a stereoisomer, a tautomer, an N-oxide, a solvate, a metabolite, a pharmaceutically acceptable salt or a prodrug thereof, for use in treating, preventing, or lessening Postoperative Delirium (POD) in a subject in need thereof,

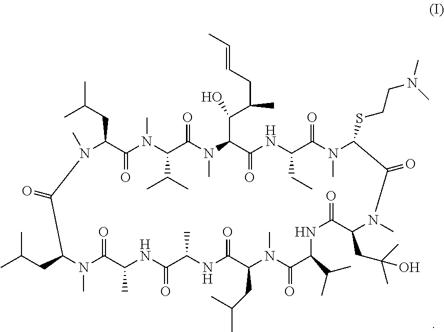 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

In one embodiment, the compound is administered to the subject within 0.5 hour prior to the operation.

In one embodiment, wherein the compound is administered at a daily dose of less than about 900 mg.

In one embodiment, wherein the compound is administered at a daily dose of between about 10 to about 900 mg.

In one embodiment, wherein the compound is administered at a daily dose of between about 50 to about 600 mg.

In one embodiment, wherein the compound is administered 1 time per day.

In one embodiment, wherein the compound is administered 1 time per day as a single dosage.

In one embodiment, wherein the compound is administered by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intraperitoneally and intrathecally.

In one embodiment, wherein the compound is administered orally or intravenously.

In one embodiment, wherein the compound is administered in a form of tablet, capsule or injection.

In one embodiment, wherein the compound is administered in combination with one or more other agent used for preventing, treating or lessening cognitive impairment other than the compound of Formula I.

Any embodiment disclosed herein can be combined with other embodiments as long as they are not contradictory to one another, even though the embodiments are described under different aspects of the invention. In addition, any technical feature in one embodiment can be applied to the corresponding technical feature in other embodiments as long as they are not contradictory to one another, even though the embodiments are described under different aspects of the invention.

The foregoing merely summarizes certain aspects disclosed herein and is not intended to be limiting in nature. These aspects and other aspects and embodiments are described more fully below.

### BRIEF DESCRIPTION OF THE FIGURES

FIG. 1 shows the diagram of the experimental design;

FIG. 2 shows the anesthesia/surgery could cause delirium-like behavior in mice (buried food test);

FIG. 3 shows the anesthesia/surgery could cause delirium-like behavior in mice (Y maze test—entries in novel arm);

FIG. 4 shows the anesthesia/surgery could cause delirium-like behavior in mice (Y maze test—duration in novel arm);

FIG. 5 shows the anesthesia/surgery could cause delirium-like behavior in mice (open field test—freezing time);

FIG. 6 shows the anesthesia/surgery could cause delirium-like behavior in mice (open field test—time spent in the center);

FIG. 7 shows the anesthesia/surgery could cause delirium-like behavior in mice (open field test—latency to center);

FIG. 8 shows the anesthesia/surgery could cause delirium-like behavior in mice (composite Z score);

FIG. 9 shows WS635 is able to attenuate the anesthesia/surgery-induced delirium-like behavior in mice (buried food test);

FIG. 10 shows WS635 is able to attenuate the anesthesia/surgery-induced delirium-like behavior in mice (Y maze test—entries in novel arm);

FIG. 11 shows WS635 is able to attenuate the anesthesia/surgery-induced delirium-like behavior in mice (Y maze test—duration in novel arm);

FIG. 12 shows WS635 is able to attenuate the anesthesia/surgery-induced delirium-like behavior in mice (open field test—freezing time);

FIG. 13 shows WS635 is able to attenuate the anesthesia/surgery-induced delirium-like behavior in mice (open field test—time spent in the center);

FIG. 14 shows WS635 is able to attenuate the anesthesia/surgery-induced delirium-like behavior in mice (open field test—latency to center);

FIG. 15 shows WS635 is able to attenuate the anesthesia/surgery-induced delirium-like behavior in mice (composite Z score).

### DESCRIPTION OF THE DISCLOSURE

### Definitions and General Terminology

Reference will now be made in detail to certain embodiments of the invention, examples of which are illustrated in the accompanying structures and formulas. The invention is intended to cover all alternatives, modifications, and equivalents which may be included within the scope of the present invention as defined by the claims. One skilled in the art will recognize many methods and materials similar or equivalent to those described herein, which could be used in the practice of the present invention. The present invention is in no way limited to the methods and materials described herein. In the event that one or more of the incorporated literature, patents, and similar materials differs from or contradicts this application, including but not limited to defined terms, term usage, described techniques, or the like, this application controls.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Unless defined otherwise, all scientific and technical terms used herein have the same meaning as is commonly understood by one skilled in the art to which this invention belongs. All patents and publications referred to herein are incorporated by reference in their entirety.

The grammatical articles “a”, “an” and “the”, as used herein, are intended to include “at least one” or “one or more” unless otherwise indicated herein or clearly contradicted by the context. Thus, the articles used herein refer to one or more than one (i.e. at least one) of the grammatical objects of the article. For example, “an embodiment” refers to one or more embodiments.

The term “comprise” is an open expression, it means comprising the contents disclosed herein, but don't exclude other contents.

The term “pharmaceutically acceptable,” as used herein, refers to those compounds, materials, compositions, and/or dosage forms which are, within the scope of sound medical judgment, suitable for use in contact with the tissues of patients without excessive toxicity, irritation, allergic response, or other problem or complication commensurate with a reasonable benefit/risk ratio, and are effective for their intended use.

The term “prodrug” refers to a compound that is transformed in vivo into a compound of Formula (I). Such a transformation can be affected, for example, by hydrolysis of the prodrug form in blood or enzymatic transformation to the parent form in blood or tissue. Prodrugs of the compounds disclosed herein may be, for example, esters. Some common esters which have been utilized as prodrugs are phenyl esters, aliphatic (C1-24) esters, acyloxymethyl esters, carbonates, carbamates and amino acid esters. For example, a compound disclosed herein that contains a hydroxy group may be acylated at this position in its prodrug form. Other prodrug forms include phosphates, such as, those phosphate compounds derived from the phosphonation of a hydroxy group on the parent compound. A thorough discussion of prodrugs is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, J. Rautio et al., Prodrugs: Design and Clinical Applications, Nature Review Drug Discovery; 2008, 7, 255-270, and S. J. Hecker et al., Prodrugs of Phosphates and Phosphonates, Journal of Medicinal Chemistry, 2008, 51, 2328-2345, each of which is incorporated herein by reference.

A “metabolite” is a product produced through metabolism in the body of a specified compound or salt thereof. The metabolites of a compound may be identified using routine techniques known in the art and their activities determined using tests such as those described herein. Such products may result for example from oxidation, reduction, hydrolysis, amidation, deamidation, esterification, deesterification, enzyme cleavage, and the like, of the administered compound. Accordingly, the invention includes metabolites of compounds disclosed herein, including metabolites produced by contacting a compound disclosed herein with a mammal for a sufficient time period.

A “pharmaceutically acceptable salts” refers to organic or inorganic salts of a compound disclosed herein. Pharmaceutically acceptable salts are well known in the art. For example, S. M. Berge et al., describe pharmaceutically acceptable salts in detail in J. Pharmaceutical Sciences, 1977, 66: 1-19, which is incorporated herein by reference. Some non-limiting examples of pharmaceutically acceptable and nontoxic salts include salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid and malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, laurylsulfate, malate, methanesulfonate, 2-naphthalenesulfonate, nicotinate, nitrate, oleate, palmitate, pamoate, pectinate, persulfate, 3-phenylpropionate, picrate, pivalate, propionate, stearate, thiocyanate, p-toluenesulfonate, undecanoate, valerate, and the like. Salts derived from appropriate bases include alkali metal, alkaline earth metal, ammonium and N⁺(C₁₋₄ alkyl)₄ salts. This invention also envisions the quaternization of any basic nitrogen-containing groups of the compounds disclosed herein. Water or oil soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counter ions such as halide, hydroxide, carboxylate, sulfate, phosphate, nitrate, C₁₋₈ sulfonate or aryl sulfonate.

The term “solvate” refers to an association or complex of one or more solvent molecules and a compound disclosed herein. Examples of solvents that form solvates include, but are not limited to, water, isopropanol, ethanol, methanol, DMSO, ethyl acetate, acetic acid, ethanolamine and the mixture thereof. The term “hydrate” refers to the complex where the solvent molecule is water.

The term “hydrate” can be used when said solvent is water. In one embodiment, one water molecule is associated with one molecule of the compounds disclosed herein, such as a hydrate. In another embodiment, more than one water molecule may be associated with one molecule of the compounds disclosed herein, such as a dihydrate. In still another embodiment, less than one water molecule may be associated with one molecule of the compounds disclosed herein, such as a hemihydrate. Furthermore, all the solvates of the invention retain the biological effectiveness of the non-hydrate form of the compounds disclosed herein.

As used herein, the term “therapeutically effective amount” or “therapeutically effective dose” refers to the amount of compound disclosed herein that can elicit the biological or medical response (such as reducing or inhibiting the activity of an enzyme or protein, or improving symptoms, lessening disorders, slowing or delaying the development of diseases and the like).

### Pharmaceutical Composition of the Compound and Preparations and Administration

In one aspect, provided herein is a pharmaceutical composition including compound of Formula (I) or a stereoisomer, a tautomer, an N-oxide, a solvate, a metabolite, a pharmaceutically acceptable salt or a prodrug thereof. The pharmaceutical compositions further comprise at least a pharmaceutically acceptable carrier, an adjuvant, or an excipient, and optionally other therapeutic and/or prophylactic ingredients.

Suitable carriers, adjuvants and excipients are well known to those skilled in the art and described in detail in such as Ansel H. C. et al., Ansel's Pharmaceutical Dosage Forms and Drug Delivery Systems (2004) Lippincott, Williams & Wilkins, Philadelphia; Gennaro A. R. et al., Remington: The Science and Practice of Pharmacy (2000) Lippincott, Williams & Wilkins, Philadelphia; and Rowe R. C., Handbook of Pharmaceutical Excipients (2005) Pharmaceutical Press, Chicago.

“Pharmaceutically acceptable excipient” as used herein means a pharmaceutically acceptable material, composition or vehicle involved in giving form or consistency to the pharmaceutical composition. Each excipient must be compatible with the other ingredients of the pharmaceutical composition when commingled, such that interactions which would substantially reduce the efficacy of the compound of the invention when administered to a patient and would result in pharmaceutically unacceptable compositions are avoided. In addition, each excipient must of course be of sufficiently high purity to render it pharmaceutically acceptable.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting the compound of the present invention once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, humectants, chelating agents, plasticizers, viscosity increasing agents, antioxidants, preservatives, stabilizers, surfactants, and buffering agents. One skilled in the art will appreciate that certain pharmaceutically acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

In Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York, the contents of each of which is incorporated by reference herein, are disclosed various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention.

In certain embodiments, the subject at risk of POD is a child or an elderly. In various embodiments, the subject is a mammal, e.g., a human.

In certain embodiments, the subject at risk of POD is an elderly.

The compound of the invention will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols, solutions, and dry powders; and (6) topical administration such as creams, ointments, lotions, solutions, pastes, sprays, foams, and gels.

It will also be appreciated that certain of the compounds of present invention can exist in free form for treatment, or where appropriate, as a pharmaceutically acceptable derivative or a prodrug thereof. According to the present invention, a pharmaceutically acceptable derivative or a prodrug includes, but is not limited to, pharmaceutically acceptable prodrugs, salts, esters, salts of such esters, or any other adduct or derivative which upon administration to a patient in need thereof is capable of providing, directly or indirectly, a compound as otherwise described herein, or a metabolite or residue thereof.

In one embodiment, the compounds disclosed herein can be prepared to oral dosage forms. In one embodiment, the compounds disclosed herein can be prepared to inhalation dosage forms. In one embodiment, the compounds disclosed herein can be prepared to dosage forms of nasal administration. In one embodiment, the compounds disclosed herein can be prepared to transdermal dosage forms. In one embodiment, the compounds disclosed herein can be prepared to dosage forms of topical administration.

The pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting, the tablets from oxidation. Film-coated tablets are compressed tablets that are covered with a thin layer or firm of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxy groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Provided herein is a pharmaceutical composition which can be prepared to a dosage form adapted for administration to a patient by inhalation, for example as a dry powder, an aerosol, a suspension, or a solution composition. In one embodiment, the invention is directed to a dosage form adapted for administration to a patient by inhalation as a dry powder. In one embodiment, the invention is directed to a dosage form adapted for administration to a patient by inhalation as a dry powder. Dry powder compositions for delivery to the lung by inhalation typically comprise a compound disclosed herein or a pharmaceutically acceptable salt thereof as a finely divided powder together with one or more pharmaceutically-acceptable excipients as finely divided powders. Pharmaceutically-acceptable excipients particularly suited for use in dry powders are known to those skilled in the art and include lactose, starch, mannitol, and mono-, di-, and polysaccharides. The finely divided powder may be prepared by, for example, micronisation and milling. Generally, the size-reduced (eg. micronised) compound can be defined by a D₅₀ value of about 1 to 10 microns (for example as measured using laser diffraction).

Pharmaceutical compositions adapted for transdermal administration may be presented as discrete patches intended to remain in intimate contact with the epidermis of the patient for a prolonged period of time. For example, the active ingredient may be delivered from the patch by iontophoresis as generally described in Pharmaceutical Research, 3(6), 318 (1986).

Pharmaceutical compositions adapted for topical administration may be formulated as ointments, creams, suspensions, lotions, powders, solutions, pastes, gels, sprays, aerosols or oils. Ointments, creams and gels, may, for example, be formulated with an aqueous or oily base with the addition of suitable thickening and/or gelling agent and/or solvents. Such bases may thus, for example, include water and/or oil such as liquid paraffin or a vegetable oil such as arachis oil or castor oil, or a solvent such as polyethylene glycol. Thickening agents and gelling agents which may be used according to the nature of the base include soft paraffin, aluminium stearate, cetostearyl alcohol, polyethylene glycols, woolfat, beeswax, carboxypolymethylene and cellulose derivatives, and/or glyceryl monostearate and/or non-ionic emulsifying agents.

The compounds disclosed herein can also be coupled to soluble polymers as targeted medicament carriers. Such polymers may encompass polyvinylpyrrolidone, pyran copolymer, polyhydroxypropylmethacrylamidophenol, polyhydroxyethylaspartamidophenol or polyethylene oxide polylysine, substituted by palmitoyl radicals. The compounds may furthermore be coupled to a class of biodegradable polymers which are suitable for achieving controlled release of a medicament, for example polylactic acid, poly-epsilon-caprolactone, polyhydroxybutyric acid, polyorthoesters, polyacetals, polydihydroxypyrans, polycyanoacrylates and crosslinked or amphipathic block copolymers of hydrogels.

The pharmaceutical compositions provided herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

The pharmaceutical compositions provided herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

The pharmaceutical compositions provided herein may be formulated as immediate or modified release dosage forms, including delayed-, sustained, pulsed-, controlled, targeted-, and programmed-release foul's.

The pharmaceutical compositions provided herein may be formulated for single or multiple dosage administration. The single dosage formulations are packaged in an ampoule, a vial, or a syringe. The multiple dosage parenteral formulations must contain an antimicrobial agent at bacteriostatic or fungistatic concentrations. All parenteral formulations must be sterile, as known and practiced in the art.

The pharmaceutical compositions provided herein may be co-formulated with other active ingredients which do not impair the desired therapeutic action, or with substances that supplement the desired action.

In one embodiment, the therapeutic methods disclosed herein comprise administrating to a patient in need of the treatment a safe and effective amount of the compound of the invention or the pharmaceutical composition containing the compound of the invention. Each example disclosed herein comprises treating the above disorders or diseases by administrating to a patient in need of the treatment a safe and effective amount of the compound of the invention or the pharmaceutical composition containing the compound of the invention.

In one embodiment, the compound of the invention or the pharmaceutical composition thereof may be administered by any suitable route of administration, including both systemic administration and topical administration. Systemic administration includes oral administration, parenteral administration, transdermal administration and rectal administration. Parenteral administration refers to routes of administration other than enteral or transdermal, and is typically by injection or infusion. Parenteral administration includes intravenous, intramuscular, and subcutaneous injection or infusion. Topical administration includes application to the skin as well as intraocular, otic, intravaginal, inhaled and intranasal administration. In one embodiment, the compound of the invention or the pharmaceutical composition thereof may be administered orally. In one embodiment, the compound of the invention or the pharmaceutical composition thereof may be administered by inhalation. In a further embodiment, the compound of the invention or the pharmaceutical composition thereof may be administered intranasally.

In one embodiment, the compound of the invention or the pharmaceutical composition thereof may be administered once or according to a dosing regimen wherein a number of doses are administered at varying intervals of time for a given period of time. For example, doses may be administered one, two, three, or four times per day. In one embodiment, a dose is administered once per day. In a further embodiment, a dose is administered twice per day. Doses may be administered until the desired therapeutic effect is achieved or indefinitely to maintain the desired therapeutic effect. Suitable dosing regimens for the compound of the invention or the pharmaceutical composition thereof depend on the pharmacokinetic properties of that compound, such as absorption, distribution, and half-life, which can be determined by the skilled artisan. In addition, suitable dosing regimens, including the duration such regimens are administered, for the compound of the invention or the pharmaceutical composition thereof depend on the disorder being treated, the severity of the disorder being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

The compounds of the present invention may be administered either simultaneously with, or before or after, one or more other therapeutic agents. The compounds of the present invention may be administered separately, by the same or different route of administration, or together in the same pharmaceutical composition as the other agents.

Compounds provided herein can used in combination with sedative, hypnotic, anxiolytic, antipsychotic, antianxiety agent, cyclopyrrolidone, imidazopyridine, pyrazolopyrimidines, minor tranquilizer, melatonin agonist and antagonist, melatoninergic agent, benzodiazepine, barbiturate, antagonist, and the like. For example: adinazolan, allobarbital, alonimid, alprazolam, amitriptyline, amobarbital, amoxapine, bentazepam, tacitin, brotizolam, bupropion, buspirone, butabarbital, butalbital, capuride, carbocloral, chloral betaine, chloral hydrate, chlorodyne, clomipramine, clonazepam, domperidone, methaminodiazepoxide, cloretate, clozapine, cyprazepam, desipramine, dexclamo, diazepam, chloralsalicylamide, divalproic acid, diphenhydramine, doxepin, estazolam, ethchlorvynol, etomidate, fenobam, flunitrazepam, flurazepam, fluvoxamine, fluoxetine, fosazepam, glutethimide, halazepam, hydroxyzine, imipramine, lithium, orazepam, lormetazepam, maprotiline, mecloqualone, melatonin, methylphenobarbital, meprobamate, methaqualone, midaflur, midazolam, nefazodone, nisobamate, nitrazepam, nortriptyline, oxezepam, paraaldehyde, paroxetine, pentobarbital, perlapine, perphenazine, phenelzine, phenobarbital, Prazepam, promethazine, isopropylphenol, protriptyline, quazepam, reclazepam, rolipram, secobarbital, sertraline, suproclone, temazepam, thioridazine, tracazolate, tranylcypromine, trazodone, triazole benzodiazepine, trepipam, tricetamide, trichloroethyl phosphate, trifluoperazine, trimetozine, trimeprimine, uldazepam, venlafaxine, zaleplon, zolazepam, zolpidem and the salts and compositions thereof, and the like. Alternatively, physical methods such as light therapy or electrical stimulation can be used during administration of compounds disclosed herein.

Additionally, the compounds of Formula (I) may be administered as prodrugs. As used herein, a “prodrug” of a compound of the invention is a functional derivative of the compound which, upon administration to a patient, eventually liberates the compound of the invention in vivo. Administration of a compound of the invention as a prodrug may enable the skilled artisan to do one or more of the following: (a) modify the onset of action of the compound in vivo; (b) modify the duration of action of the compound in vivo; (c) modify the transportation or distribution of the compound in vivo; (d) modify the solubility of the compound in vivo; and (e) overcome a side effect or other difficulty encountered with the compound. Typical functional derivatives used to prepare prodrugs include modifications of the compound that are chemically or enzymatically cleaved in vivo. Such modifications, which include the preparation of phosphates, amides, esters, thioesters, carbonates, and carbamates, are well known to those skilled in the art.

### Use of the Compounds and Pharmaceutical Compositions

Compounds WS635 or pharmaceutical compositions disclosed herein are efficient for treating or preventing POD.

An “effective amount”, “a therapeutically effective amount” or “effective dose” of the compound, or a stereoisomer, a tautomer, an N-oxide, a solvate, a metabolite, a pharmaceutically acceptable salt or a prodrug thereof or pharmaceutically acceptable composition disclosed herein is an amount that is effective in treating or lessening the severity of postoperative cognitive dysfunction. The complex and pharmaceutically acceptable compositions are effective administered in a fairly wide dose range. For example, the daily dose is from about 0.1 mg to 1000 mg per person, the compounds or pharmaceutically acceptable compositions can be administered in a single dose or in several divided doses a day. The compound and compositions, according to the method disclosed herein, may be administered using any amount and any route of administration which is effective for treating or lessening the severity of the postoperative cognitive dysfunction. The exact amount required will vary from subject to subject, depending on the species, age, and general condition of the subject, the severity of the infection, the particular agent, its mode of administration, and the like. The compound or pharmaceutical composition disclosed herein can also be administered with one or more other therapeutic agents as discussed above.

In some embodiments, the compound described herein can be administered to a subject within 1 hour prior to the operation or within 0.5 hour prior to the operation. The compound described herein can be administered at a daily dose of less than about 900 mg, for example, at a daily dose of between about 10 to about 900 mg or at a daily dose of between about 50 to about 600 mg. The compound can be administered 1 time per day or can be administered 1 time per day as a single dosage.

Besides being useful for human treatment, WS635 and the compositions thereof are also useful for veterinary treatment of animals such as companion animals, exotic animals and mammals of farm animals. In other embodiments, the animals disclosed herein include horses, dogs, and cats. As used herein, the compounds disclosed herein include the pharmaceutically acceptable derivatives thereof.

### Examples

Materials and Methods

Mice Anesthesia and Surgery. 18-month-old C57BL/J6 female mice were adaptive in lab for 3 days. The mice were randomly assigned to the anesthesia/surgery group or control group by weight. The mice in the anesthesia/surgery group had a simple laparotomy under isoflurane anesthesia using the methods described in our previous studies. Specifically, we anesthetized each of the mice using 1.4% isoflurane in 100% oxygen in a transparent acrylic chamber. Fifteen minutes after the induction, we moved the mouse out of the chamber. Isoflurane anesthesia was maintained via a cone device, and one 16-gauge needle was inserted into the cone near the nose of the mouse to monitor the concentration of isoflurane. We made a longitudinal midline incision from the xiphoid to the 0.5 centimeter proximal pubic symphysis on the skin, abdominal muscles and peritoneum. We then sutured the incision layer by layer with 5-0 Vicryl thread.

Mice Anesthesia for isoflurane-induced cognitive impairment experiment. 8 month-old C57BL/6J mice. Anesthesia: 1.4% isoflurane for two hours.

WS635 Treatment for isoflurane-induced cognitive impairment experiment. WS635 was dissolved in 10% DMSO plus corn oil. Each mouse received 26.4 mg/kg WS635 or same volume of vehicle (10% DMSO plus corn oil) at 30 minutes before the anesthesia. Then, the mice were tested in Fear Conditioning System at 48 hours and 7 days after the anesthesia.

WS635 Treatment. WS635 was dissolved in corn oil with 10% DMSO, each of the mice will be injected with WS635 solution in the dose of 40 mg/kg for 0.2 ml through IP at 30 minutes before control condition or anesthesia/surgery by using 27G×½ needles. The mice in control group received 0.2 ml of corn oil with 10% DMSO.

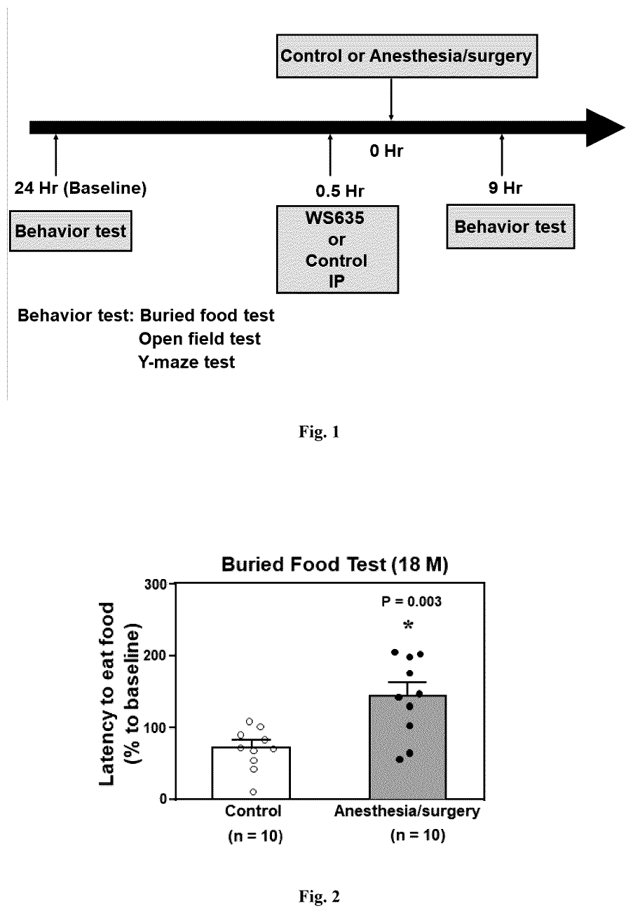

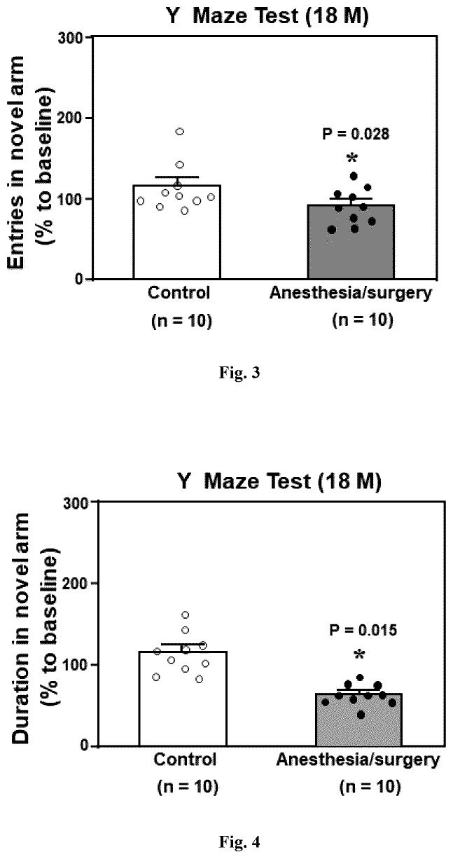

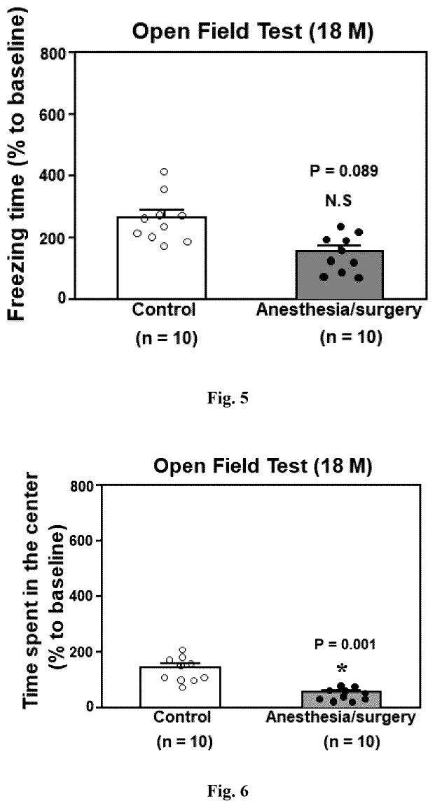

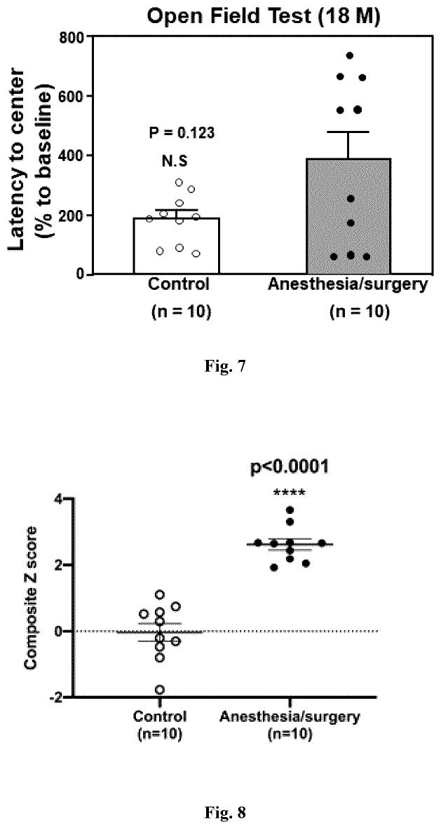

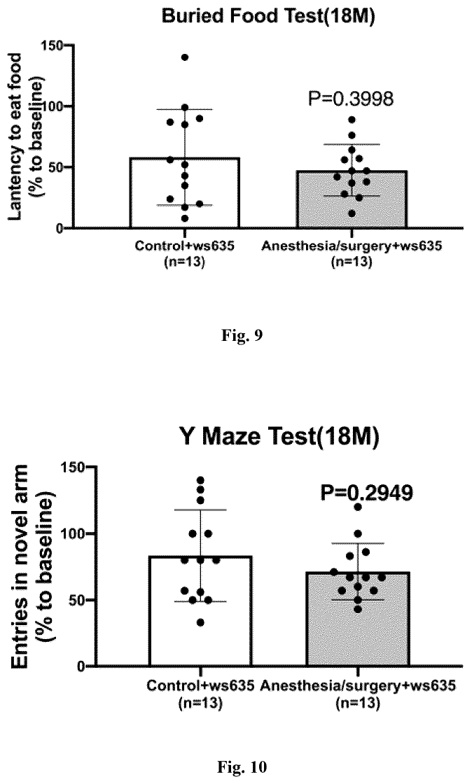

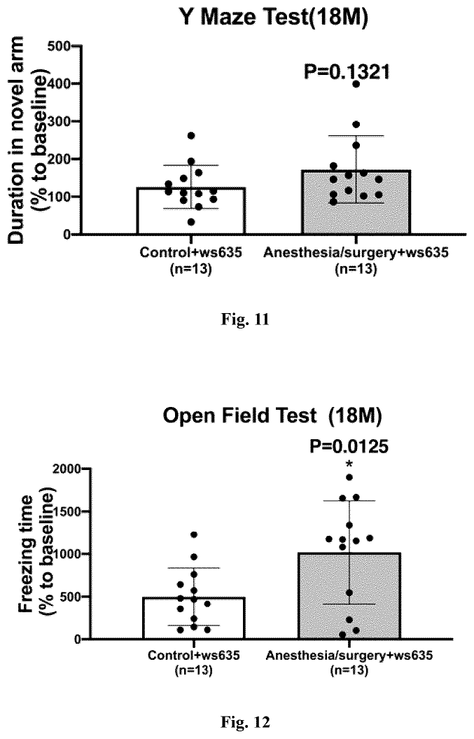

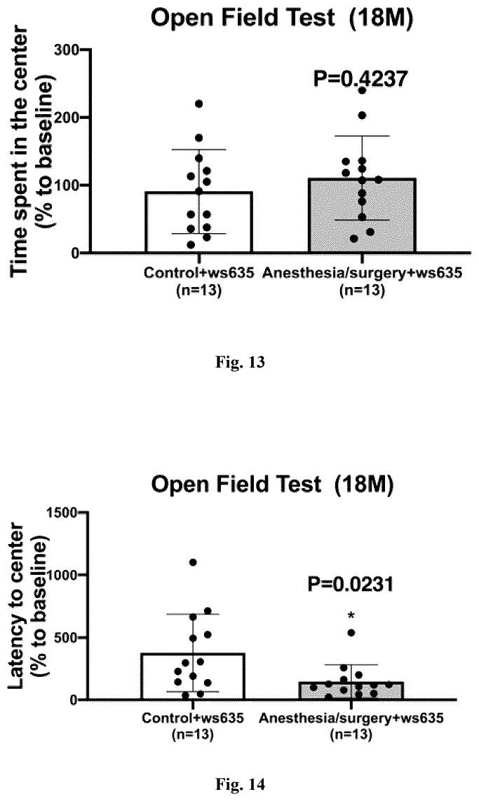

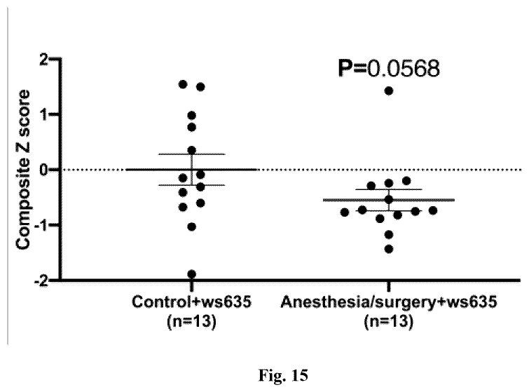

Behaviors Tests. As demonstrated in the diagram (FIG. 1), all mice will have multiple behavioral tests in the order of buried food test, then open field test and finally Y maze test at 24 hours before (baseline) the anesthesia/surgery or control condition, and at 6, 9, and 24 hours after the Anesthesia/Surgery. We will perform the behavior tests in groups of 3 mice and finished them within 50 minutes, which mimics the certain features of clinical evaluation of delirium in patients.

Buried Food Test. Specifically, two days before the test, we will give each mouse 2 pieces of the sweetened cereal. On all test days, we will habituate the mice for one hour prior to the test by placing the home cage with mice in the testing room. The test cage will be prepared with clean bedding (3 centimeters high). We will bury 1 sweetened cereal pellet 0.5 centimeter below the surface of bedding so that it will be invisible. The location of the food pellet will be changed every time in a random fashion. We will place the mouse in the center of the test cage and measure the latency of the mouse to eat the food. Latency will be defined as the time from when the mouse is placed in the test cage until when the mouse uncovers the food pellet and grasps it in the forepaws and/or teeth. Mice will be allowed to consume the pellet they find and will be then returned to their home cage. The observation time will be 5 minutes. If the mouse cannot find the pellet within 5 minutes, the testing session will end and the latency will be defined as 300 seconds for that mouse. We will empty the bedding from the test cage and clean the cage with 70% ethanol solution after each test to prevent the transmission of olfactory cues. We will change gloves after each test.

Open Field Test. Specifically, the mouse will be gently placed in the center of an open field chamber (40×40×40 centimeters) under dim light and will be allowed to move freely for 5 minutes. The movement parameters of the mouse will be monitored and analyzed via a video camera connected to the Any-Maze animal tracking system software (Stoelting Co., Wood Dale, IL). The total distance moved (meters), the time (seconds) spent in the center of the open field, the freezing time (seconds) and the latency (the time in seconds for the mice to reach to the location at the first attempt) to the center of the open field will be recorded and analyzed. The floor of the open field will be cleaned with 70% ethanol solution between each test.

Y Maze Test. Specifically, the Y maze, made of gray polyvinylene, will be placed in a quiet and illuminated room. Each maze will consist of three arms (8×30×15 centimeters, width×length×height), with an angle of 120 degrees between each arm. The three arms will includes the start arm, in which the mouse will start to explore (always open); the novel arm, which will be blocked at the first trial, but opened at the second trial; and the other arm (will always open). In the experiment, the start arm and other arm will be designed randomly to avoid spatial memory error. The Y maze test will consist of 2 trials separated by an inter-trial interval (ITI). The first trial (training) will be 10 minutes in duration, which will allow the mouse to explore 2 arms (the start arm and other arm) of the maze, with the novel arm being blocked. After a 2 hours (for the studies of 6 and 24 hours after the Anesthesia/Surgery) or 4 hours (for the studies of 9 hours after the Anesthesia Surgery) ITI, the second trial (retention) will be conducted. For the second trial, the mouse will be placed back in the maze in the same start arm with free access to all 3 arms for minutes. A video camera, which will be linked to the Any-Maze animal tracking system software, will be installed 60 centimeters above the chamber to monitor and analyze the number of entries and the time spent in each arm. The time spent in and entries into the novel arms will indicate the spatial recognition memory (learned behavior). Each of the arms of the Y maze will be cleaned with 70% ethanol solution between trials.

Results

Diagram of the Experimental Design is Shown in Figure/the mice will receive behavior tests at 24 hours (baseline) before the abdominal surgery under isoflurane anesthesia (anesthesia/surgery), and then at 9 hours after the anesthesia/surgery.

WS635 can Attenuate the Anesthesia/Surgery-Induced Changes of Mice in Buried Food Test.

The anesthesia/surgery significantly increases the latency to eat food of the mice in the buried food test as compared to the control condition. The treatment with 40 mg/kg WS635 can attenuate the anesthesia/surgery-induced changes of mice in these delirium-like behaviors.

WS635 can Attenuate the Anesthesia/Surgery-Induced Changes of Mice in Y Maze Test.

The anesthesia/surgery significantly decreases the number of entries in the novel arm in the Y maze test as compared to the control condition. The anesthesia/surgery significantly decreases the duration in the novel arm in the Y maze test as compared to the control condition. The treatment with 40 mg/kg WS635 can attenuate the anesthesia/surgery-induced changes of mice in these delirium-like behaviors.

WS635 can Attenuate the Anesthesia/Surgery-Induced Changes of Mice in Open Field Test.

The anesthesia/surgery significantly decreases the freezing time in the open field test as compared to the control condition. The anesthesia/surgery significantly decreases the time spent in the center of the open field as compared to the control condition. The anesthesia/surgery significantly increases the latency to the center in the open field test as compared to the control condition. The treatment with 40 mg/kg WS635 can attenuate the anesthesia/surgery-induced changes of mice in these delirium-like behaviors.

Reference throughout this specification to “an embodiment,” “some embodiments,” “one embodiment”, “another example,” “an example,” “a specific examples,” or “some examples,” means that a particular feature, structure, material, or characteristic described in connection with the embodiment or example is included in at least one embodiment or example of the present disclosure. Thus, the appearances of the phrases such as “in some embodiments,” “in one embodiment”, “in an embodiment”, “in another example, “in an example,” “in a specific examples,” or “in some examples,” in various places throughout this specification are not necessarily referring to the same embodiment or example of the present disclosure. Furthermore, the particular features, structures, materials, or characteristics may be combined in any suitable manner in one or more embodiments or examples.

What is claimed is:

1. A method for preventing, treating or lessening Postoperative Delirium (POD) in a patient comprising administering to the patient therapeutically effective amount of the compound having Formula (I) or a stereoisomer, a tautomer, an N-oxide, a solvate, a metabolite, a pharmaceutically acceptable salt or a prodrug thereof,

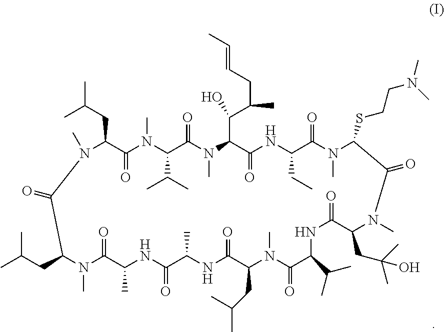 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

2. The method of claim 1, wherein the therapeutically effective amount is 40 mg/kg in mice.

3. The method of claim 1, wherein the therapeutically effective amount is 4.4 mg/kg in human.

4. The method of claim 1, wherein the compound is administered to the subject within 1 hour, and preferably, 0.5 hour, prior to the operation.

5. The method of claim 1, wherein the compound is administered at a daily dose of less than about 900 mg, preferably, between about 10 to about 900 mg, and more preferably, between about 50 to about 600 mg.

6. The method of claim 1, wherein the compound is administered 1 time per day.

7. The method of claim 1, wherein the compound is administered by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intraperitoneally and intrathecally, and
preferably, the compound is administered orally, intravenously or intraperitoneally.

8. The method of claim 1, wherein the compound is administered in a form of tablet, capsule or injection.

9. The method of claim 1, wherein the compound is administered in combination with one or more other agent used for preventing, treating or lessening cognitive impairment other than the compound of Formula I.

10. A pharmaceutical composition comprising a therapeutically effective amount of a compound of Formula I or a stereoisomer, a tautomer, an N-oxide, a solvate, a metabolite, a pharmaceutically acceptable salt or a prodrug thereof, for use in treating, preventing, or lessening Postoperative Delirium (POD),

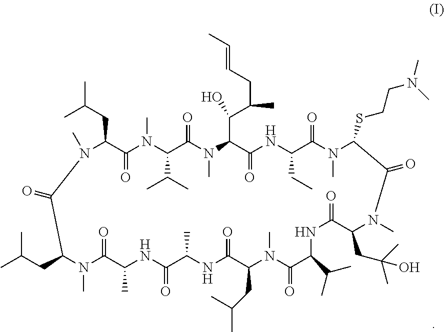 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

11. The pharmaceutical composition of claim 10, wherein the composition is formulated in a single dose form wherein such single dose form comprises less than 900 mg of Compound I.

12. The pharmaceutical composition of claim 11, wherein such single dose further comprise one or more other agent used for preventing, treating or lessening cognitive impairment other than the compound of Formula I.

13. A compound of Formula I or a stereoisomer, a tautomer, an N-oxide, a solvate, a metabolite, a pharmaceutically acceptable salt or a prodrug thereof, for use in treating, preventing, or lessening Postoperative Delirium (POD) in a subject in need thereof,

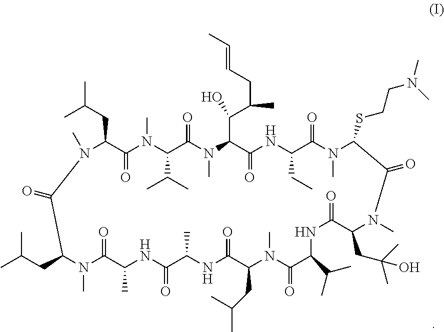 [SMILES: C/C=C/C[C@@H](C)[C@@H](O)[C@H]1C(=O)N[C@@H](CC)C(=O)N(C)[C@H](SCCN(C)C)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

14. The compound of claim 13, wherein the compound is administered to the subject within hour prior to the operation.

15. The compound of claim 13, wherein the compound is administered at a daily dose of less than about 900 mg, preferably, between about 10 to about 900 mg, and more preferably, between about 50 to about 600 mg.

16. The compound of claim 13, wherein the compound is administered 1 time per day as a single dosage.

17. The compound of claim 13, wherein the compound is administered by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intraperitoneally and intrathecally.

18. The compound of claim 13, wherein the compound is administered orally or intravenously.

19. The compound of claim 13, wherein the compound is administered in a form of tablet, capsule or injection.

20. The compound of claim 13, wherein the compound is administered in combination with one or more other agent used for preventing, treating or lessening cognitive impairment other than the compound of Formula I.
